(19) United States
(12) Plant Patent Application Publication
(10) Pub. No.: US 2009/0255019 P1
(43) Pub. Date: Oct. 8, 2009

(54) Hydrangea plant named 'Glory"

(50) ;

(76) Inventor: Harrison Higaki, San Mateo, CA (US)

Correspondence Address: JAMES R. CYPHER, 405 14TH STREET, SUITE 1607, OAKLAND, CA, 94612, US

(21) Appl. No.: 12/082,145

(22) Filed: Apr. 8, 2008

(51) Int. Cl. A01H 5/00

(52) U.S. Cl. PLT/250

(57) ABSTRACT

A new and distinct cultivar of Hydrangea macrophylla (Thunb.) named ‘Glory’ originated as a controlled cross. The cultivar ‘Glory’ has non-pigmented sepalous florets and is desirable for its compact and sturdy growth habit. ‘Glory’ has large showy inflorescences, having both sepalous and non-sepalous florets, dominated non-sepalous florets.

### BOTANICAL CLASSIFICATION

Hydrangea macrophylla (Thunb.) ‘Glory’

### VARIETY DENOMINATION

‘Glory’

### BACKGROUND OF THE INVENTION

This invention relates to a new and distinct cultivar of the Saxifragaceae family. The botanical name of the plant is Hydrangea macrophylla (Thunb.) ‘Glory’.

The new cultivar originated as a seedling from a controlled cross.

The variety ‘Glory’ has non-pigmented sepals. The new variety was first noticed, because it combined the desirable growth characteristics of its parents while producing flowers with non-pigmented sepals.

The new cultivar ‘Glory’ has been successfully asexually reproduced under controlled environmental conditions at a nursery in Half Moon Bay, Calif. under the direction of the inventor over a three year period with its distinguishing characteristics remaining stable.

Asexual reproduction was first accomplished when vegetative cuttings were taken from the initially selected plant. Examination of asexually reproduced, successive generations grown in at Half Moon Bay, Calif. show that the combination of characteristics as herein disclosed for ‘Fantasy’ remains firmly fixed through three generations.

### DESCRIPTION OF THE DRAWINGS

The accompanying drawings consist of color photographs that show the typical plant form, including the inflorescence, foliage, and sepals.

FIG. 1 is a view of the entire plant showing its form, compact growth habit, dark green foliage, inflorescence, and non-pigmented sepals.

FIG. 2 is a close-up view of a panicle of the new variety, showing opened, sepalous florets.

FIG. 3 is a top view of the new variety.

FIG. 4 is a close-up view of the adaxial surface of a mature leaf.

FIG. 5 is a close-up view of a non-woody portion of the stem, showing lenticels on the stem.

FIG. 6 a view of a non-woody portion of the stem, showing lenticels on the stem

FIG. 7 is a close-up view of the aBaxial surface of a mature leaf.

FIG. 8 is a close-up view of the woody portion of the stem.

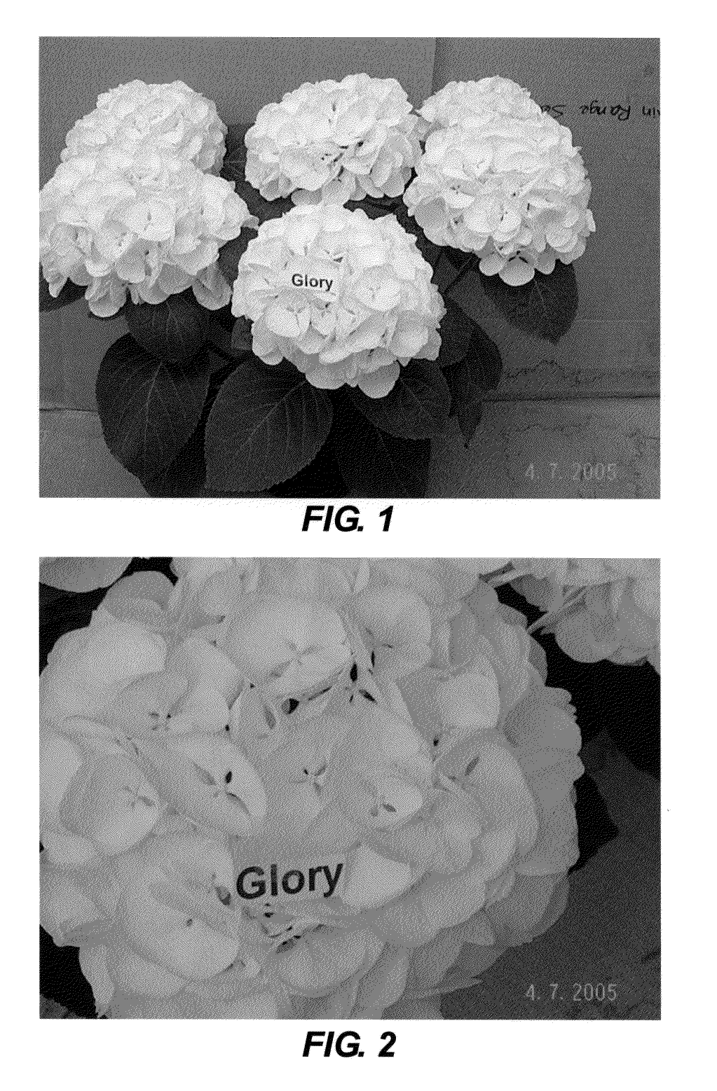

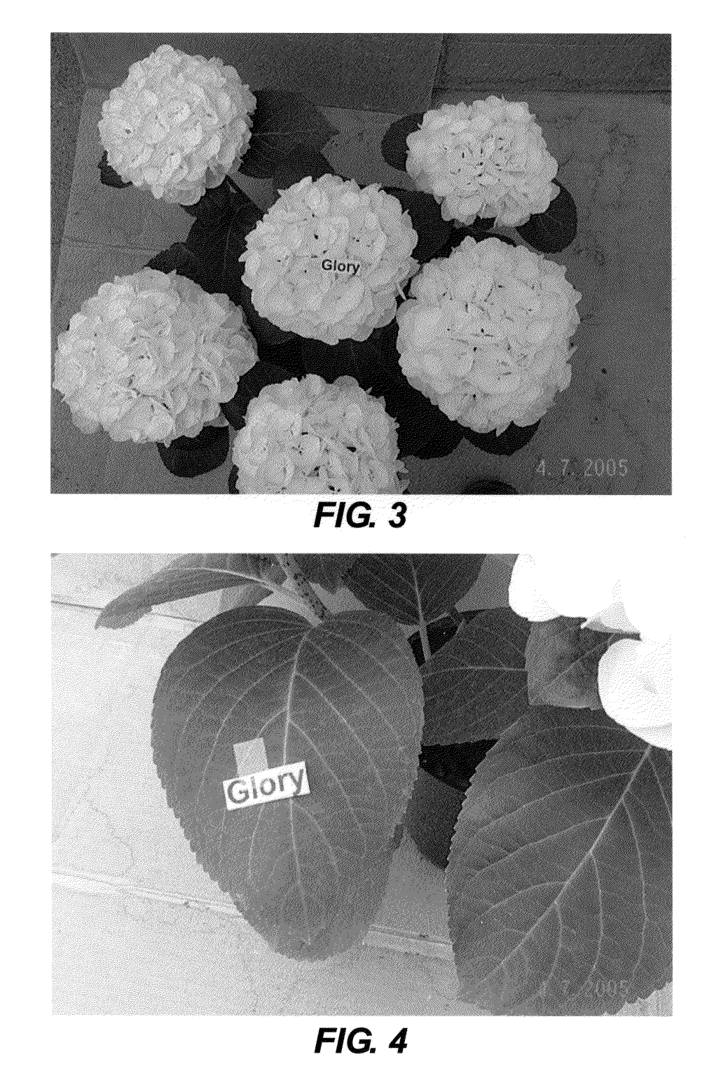

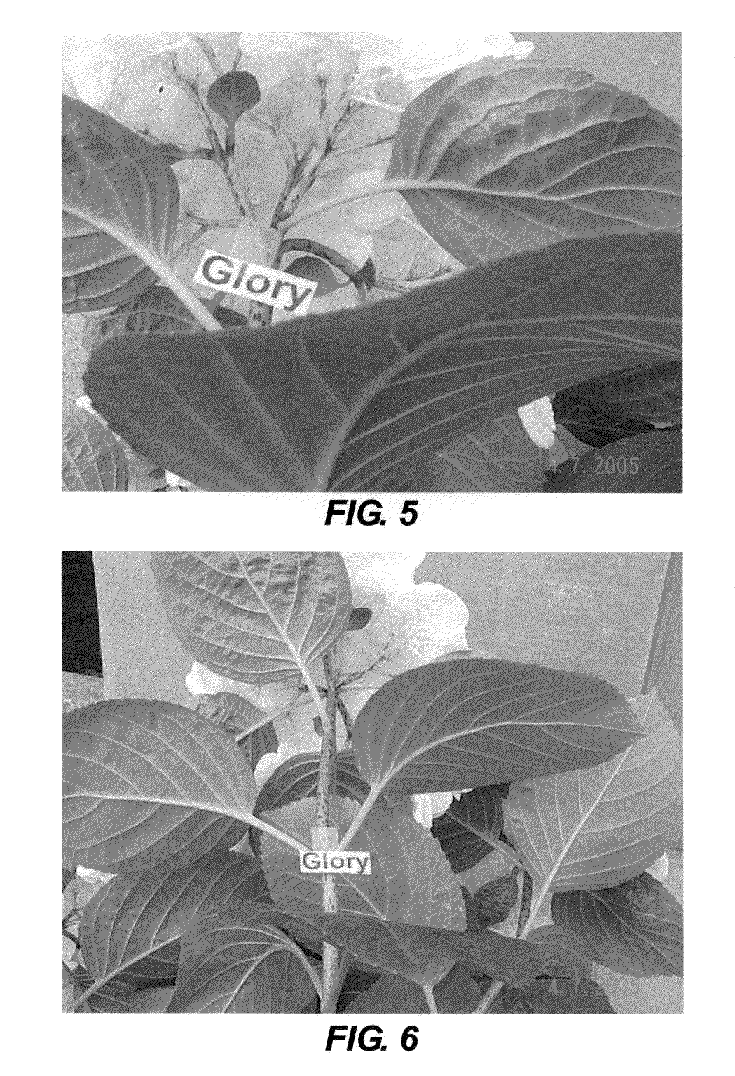

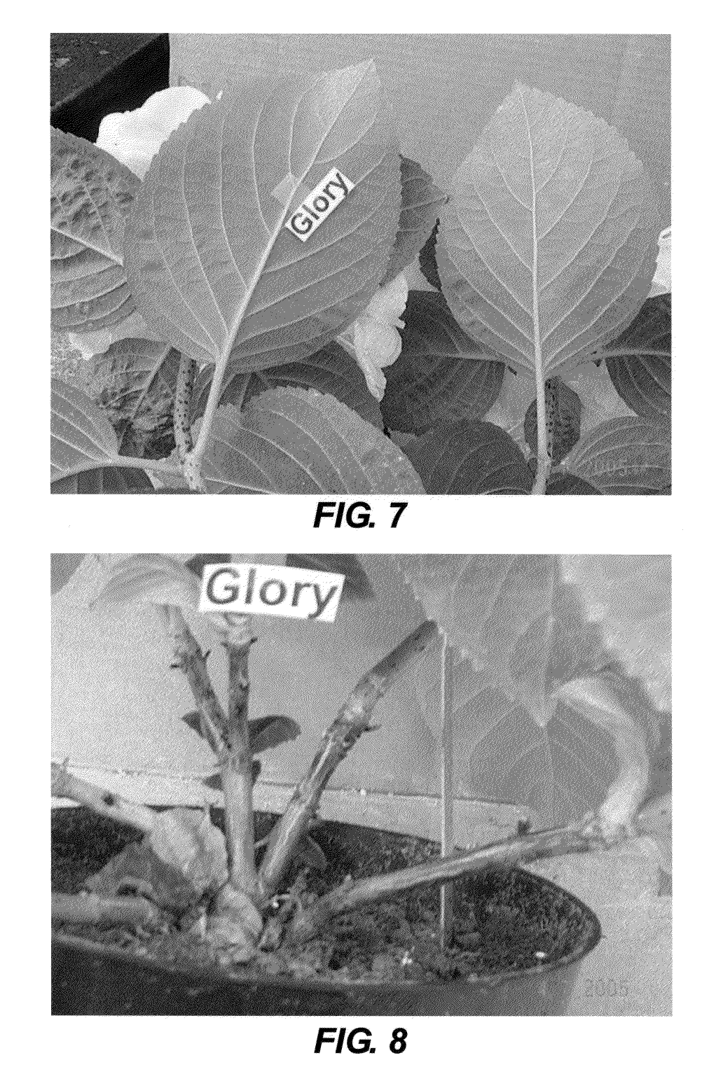

### DESCRIPTION OF THE NEW PLANT

The plants shown in the figures are approximately 1 years old. The plant started out as cuttings, taken from the stem of a grown plant.

‘Glory’ has not been observed under all possible environmental conditions. The phenotype may vary significantly with variations in environment such as temperature, light intensity and day length. Color determinations were made with The Royal Horticultural Society (R.H.S.) Colour Chart.
The plant:
   Origin.—Controlled cross.
   Form.—Upright, compact shrub. A typical plant with a mature inflorescence that is ready for sale is approximately 17″ high and has a diameter of 16″ when grown in a 4″ pot with appropriate soil amendments.
   Growth.—Upright, vigorous growth habit. Inflorescence is large. The plant branches easily with shoots forming at the base of the plant. Lateral branches are similar in appearance and form to the main stems.
   Stems.—Lenticels are present. Lenticels are R.H.S. 59B red-purple group. Stems become woody as they age. The color of typical young stems and young lateral branches is R.H.S. 145 A (green group).
   Foliage.—Abundant. Leaves are opposite on stem and lateral branches.
   Shape of leaf.—Elliptic with acute base and apex. Margins are serrate.
   Texture.—Glabrous; veins dominate on the underside of the leaf and are sunken on the leaf surface.
   Color.—Mature leaves have an upper side that is R.H.S. 147 A (yellow-green group), and an under side that is R.H.S. 138 B (green group). Leaves are pinnately veined. The midvein and veins branching off the midvein are large and prominent on the underside of the leaves. Leaves are as wide as 5.75″ and 7.25″ long. Petioles are typically 1″ to 1.5″ long.
Buds:
   Form.—Globose with 4 to 5 connate petals. The majority of buds have sepals. Buds are approximately 1 mm by 1 mm when very young. Buds can be 5 mm in diameter and still unopened.
   Aspect.—Smooth.
   Arrangement.—Borne on branched panicles.
Inflorescence:
   Form.—Paniculate. Terminal. As many as 50 or more individual flowers (florets) per inflorescence. Both sepalous florets and non-sepalous florets borne on same panicle. Non-sepalous florets are hidden by sepalous florets. Non-sepalous florets dominate. Flowers do not produce a fragrance. The peduncle or panicle for the inflorescence is strong and upright.
   Size of inflorescence.—Large. Individual inflorescence size is dependent on the number of florets. A typical inflorescence can grow as large as 9″ in diameter, and 5.5″ high.
   Shape.—Clusters of numerous small florets; sepalous florets are flat and overlapping one another. Sepals are persistent.
   Appearance.—Showy.
Florets:
   General.—Sepalous florets are perfect and complete.
   Corolla.—Generally there are 5 petals which fall off as flower matures. Petals are typically 6 mm long and 3 mm wide.
   Stamens.—10 stamens. Pollen is white — R.H.S. 158C (yellow-white group). Plant produces abundant pollen. Filament is approximately 2 mm long. Anther filament is 1 mm long. Anther can be tall as 4 mm and is regular and basally attached. Anther is R.H.S. 148A (yellow-green group).
   Stigma.—Two to three style each, although most florets have two style. Each style has one stigma. Style is typically 2 mm long.
   Ovary.—Ovary is partially inferior.
   Sepalous florets.—General. — Veins dominate on the underside of the sepals.
   Number of sepals.—4 or 5 sepals per floret, usually 4.
   Aspect of sepals.—Smooth and glaucescent.
   Shape of sepals.—Reniform with acuminate apex. Edges often wavy when the floret is young.
   Size of sepals.—As the florets mature, the sepals enlarge and overlap each other more and more, until, often, there is no space between the sepals when the petals of the florets open. Sepals at maturity are typically 2.5 cm long and 2 cm wide. Flowers are typically 4.5 cm in diameter.
   Fruit.—none.

I claim:

1. A new and distinct Hydrangea macrophylla plant named ‘Glory’ substantially as herein shown and described.
